(19) United States
(12) Patent Application Publication
(10) Pub. No.: US 2025/0058012 A1
(43) Pub. Date: Feb. 20, 2025

(54) INTERNAL DISINFECTION AND REMINDERS FOR THERMAL SYSTEM

(71) Applicant: Stryker Corporation, Kalamazoo, MI (US)

(72) Inventors: Marco Constant, Portage, MI (US); Christopher John Hopper, Kalamazoo, MI (US)

(21) Appl. No.: 18/724,472

(22) PCT Filed: Feb. 27, 2023
(86) PCT No.: PCT/US2023/013954

§ 371 Date: Jun. 26, 2024

Related U.S. Application Data
(60) Provisional application No. 63/315,367, filed on Mar. 1, 2022.

(51) Int. Cl. A61L 2/24; A61F 7/00; A61L 2/26; G16H 20/30; G16H 40/63

(52) U.S. Cl. CPC A61L 2/24; A61F 7/0085; A61L 2/26; G16H 20/30; G16H 40/63; A61F 2007/0054; A61F 2007/0093; A61L 2202/14; A61L 2202/24

(57) ABSTRACT

A thermal control unit for controlling a patient's temperature during a thermal therapy session is provided. The thermal control unit may include a disinfection control and/or a disinfection status indicator. When the disinfection control is activated, the thermal control unit may activate a disinfection wizard adapted display instructions for guiding a technician through a disinfection cycle. The disinfection wizard may au-tomate portions of the disinfection cycle. The thermal control unit may also send disinfection status to a server, which is adapted to forward that disinfection status to one or more electronic devices. The server may also forward reminder messages to technicians indicating when a next disinfection cycle is due. The thermal control unit itself may also indicate when a next disinfection cycle is due.

### CROSS-REFERENCE TO RELATED APPLICATIONS

This application claims priority to U.S. provisional patent application Ser. No. 63/315,367 filed Mar. 1, 2022, by inventors Marco Constant et al. and entitled INTERNAL DISINFECTION AND REMINDERS FOR THERMAL SYSTEM, the complete disclosure of which is incorporated herein by reference.

### BACKGROUND

The present disclosure relates to the internal disinfection of a thermal control system for controlling the temperature of circulating fluid that is delivered to one or more thermal pads positioned in contact with a patient.

Thermal control systems require periodic internal disinfection to ensure the fluid circulating therein does not harbor harmful bacteria. Known internal disinfection processes involve a technician using a physical copy of the instructions for use and following a detailed step by step process to prepare the thermal control unit, the hoses, the reservoir, and distilled water. The technician must run the distilled water with disinfectant through the internal water path and hoses to effectively clean the unit. Then the technician must run distilled water through the internal water path and hoses to rinse the unit. These disinfection processes involve many steps and requires the technician to intervene at multiple points, which makes the processes time-consuming and complicated to perform. Known thermal control systems are unaware of the internal disinfection process and simply operate according to the operator's directions. This means there are many potential points of human error in the internal disinfection process.

In addition to performing the internal disinfection process, known thermal control units require the technician to track the disinfection schedule for each thermal control unit. The thermal control unit must be disinfected periodically. In some cases, the thermal control unit must be disinfected a given period after its first use. The technician must record when the thermal control unit was disinfected and when it was used for the first time. Thermal control units are often stored for lengthy periods in between uses. The thermal control unit may only be removed from storage when it is needed for use on a patient. However, when the thermal control unit is removed from storage it may need to be disinfected before use, which can throw off treatment timelines.

### SUMMARY

The present disclosure is directed to a thermal control unit that includes a disinfection wizard for guiding a technician through an internal disinfection process for a thermal control system. The present disclosure is also directed to a disinfection reminder system for providing timely reminders to technicians of when a next disinfection cycle is due. Still other improved aspects of the internal disinfection and disinfection reminder system disclosed herein will be apparent to those skilled in the art in light of the following written description.

According to one embodiment of the present disclosure, a thermal control unit is provided that includes a fluid outlet, a fluid inlet, a circulation channel, a pump, a heat exchanger, a display, a clock, a disinfection control, and a controller. The thermal control unit is adapted to control a patient's temperature during a thermal therapy session. The fluid outlet is adapted to couple to a first hose in fluid communication with a thermal pad in contact with a patient, and the fluid inlet is adapted to couple to a second hose in fluid communication with the thermal pad. The circulation channel is coupled to the fluid inlet and the fluid outlet. The pump is adapted to circulate the fluid through the circulation channel from the fluid inlet to the fluid outlet. The heat exchanger is adapted to add or remove heat from the fluid circulating in the circulation channel. The disinfection control is adapted to initiate a disinfection cycle. The controller is adapted to control the heat exchanger in order to control the patient's temperature, and to display a series of disinfection steps corresponding to the disinfection cycle in response to the disinfection control being activated. The series of steps includes a particular step in which the controller automatically performs at least one of the following: (a) selects a target temperature for the circulating fluid and controls the heat exchanger such that a temperature of the fluid in the circulation channel is changed to the target temperature; or (b) measures a set amount of time and provides an indication to the user after the set amount of time has elapsed.

According to another embodiment of the present disclosure, a thermal control unit is provided that includes a fluid outlet, a fluid inlet, a circulation channel, a pump, a heat exchanger, a display, a clock, a disinfection control, and a controller. The thermal control unit is adapted to control a patient's temperature during a thermal therapy session. The fluid outlet is adapted to couple to a first hose in fluid communication with a thermal pad in contact with a patient, and the fluid inlet is adapted to couple to a second hose in fluid communication with the thermal pad. The circulation channel is coupled to the fluid inlet and the fluid outlet. The pump is adapted to circulate the fluid through the circulation channel from the fluid inlet to the fluid outlet. The heat exchanger is adapted to add or remove heat from the fluid circulating in the circulation channel. The disinfection control is adapted to initiate a disinfection cycle. The controller is adapted to control the heat exchanger in order to control the patient's temperature, and to display an indicator on the display. The indicator indicates information about a disinfection status of the thermal control unit.

According to another embodiment of the present disclosure, a disinfection reminder system is provided that includes a thermal control unit and a server. The thermal control unit includes a fluid outlet, a fluid inlet, a circulation channel, a pump, a heat exchanger, a display, a communication interface, and a controller. The thermal control unit is adapted to control a patient's temperature during a thermal therapy session. The fluid outlet is adapted to couple to a first hose in fluid communication with a thermal pad in contact with a patient, and the fluid inlet is adapted to couple to a second hose in fluid communication with the thermal pad. The circulation channel is coupled to the fluid inlet and the fluid outlet. The pump is adapted to circulate the fluid through the circulation channel from the fluid inlet to the fluid outlet. The heat exchanger is adapted to add or remove heat from the fluid circulating in the circulation channel. The controller is adapted to control the heat exchanger in order to control the patient's temperature, and to determine when a disinfection cycle has been completed for the thermal control unit. The communication interface is adapted to transmit a message to the server indicating the disinfection cycle of the thermal control unit has been completed. The server is adapted to receive the message, to compare a time of the completed disinfection cycle to a disinfection schedule, and to send a reminder message to an electronic device. The reminder message indicates that a subsequent disinfection cycle should be performed on the thermal control unit.

According to other aspects of the present disclosure, the display is part of a touch screen adapted to progress through the series of disinfection steps based on user input on the touch screen.

In some aspects, the series of disinfection steps includes a step in which the controller displays a message on the display reminding the user to drain the thermal control unit.

In some aspects, the series of disinfection steps includes a step in which the controller displays a graphical representation of the thermal control unit.

In some aspects, the controller is adapted to display a completion message on the display when the disinfection cycle is completed.

The series of disinfection steps, in some aspects, includes a step in which the controller displays on the display a graphical representation of a piece of equipment used in the disinfection process.

In some aspects, the piece of equipment is container adapted to hold at least one gallon of sterile liquid.

The series of disinfection steps, in some aspects, includes a step in which the controller displays a timer counting down from a predetermined time.

The series of disinfection steps, in some aspects, includes a step in which the controller displays instructions to the user to physically manipulate the thermal control unit.

The series of disinfection steps, in some aspects, includes a step in which the controller displays instructions to the user to place a reservoir inside the thermal control unit.

The series of disinfection steps, in some aspects, includes a step in which the controller displays instructions to the user to disconnect a hose from a port of the thermal control unit.

The series of disinfection steps, in some aspects, includes a step in which the controller displays instructions to the user to connect a hose to a hydraulic connector in a lid of a reservoir.

The series of disinfection steps, in some aspects, includes a step in which the controller is adapted to display instructions to the user to confirm whether the thermal control unit has been drained, as well as a yes indicator and a no indicator.

The controller, in some aspects, is adapted to proceed to a next disinfection step in the series of disinfection steps if the user selects the yes indicator, and to not proceed to the next step if the user selects the no indicator.

The thermal control unit, in some aspects, includes a communication interface adapted to transmit a message to a server when the disinfection cycle has been completed.

The message, in some aspects, indicates a time when the disinfection cycle was completed.

The controller, in some aspects, is adapted to display an indicator on the display, the indicator indicating when a next disinfection cycle should be performed on the thermal control unit.

In some aspects, the indicator indicates a number of days until the next disinfection cycle should be performed.

In some aspects, the indicator indicates at least one of: up to date, disinfection required soon, disinfection required, or disinfection overdue.

In some aspects, the reminder message indicates a number of days until the subsequent disinfection cycle should be performed.

The electronic device, in some aspects, is one of a smart phone, a tablet computer, a laptop computer, or a desktop computer.

In some aspects, the reminder message includes a disinfection status for the thermal control unit.

In some aspects, the controller is further adapted to determine the disinfection status based on a disinfection schedule for the thermal control unit.

The controller, in some aspects, is further adapted to display a disinfection due message on the display when the disinfection status indicates that disinfection is required or disinfection is overdue, wherein the controller is further adapted to allow a user to bypass the disinfection due message and operate the thermal control unit with performing a disinfection cycle. In other aspects, the controller is adapted to prevent a user from operating the thermal control unit without performing the disinfection cycle.

In some aspects, the disinfection status indicates at least one of: a number of days since the disinfection cycle took place; a number of days until the subsequent disinfection cycle is due; or a number of days the subsequent disinfection cycle is overdue.

The server, in some aspects, is a cloud computing server.

The electronic device, in some aspects, is adapted to display a visual indication of whether disinfection is due for the thermal control unit.

The communication interface, in some aspects, includes a Wi-Fi transceiver.

Before the various embodiments disclosed herein are explained in detail, it is to be understood that the claims are not to be limited to the details of operation or to the details of construction, nor to the arrangement of the components set forth in the following description or illustrated in the drawings. The embodiments described herein are capable of being practiced or being carried out in alternative ways not expressly disclosed herein. Also, it is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof. Further, enumeration may be used in the description of various embodiments. Unless otherwise expressly stated, the use of enumeration should not be construed as limiting the claims to any specific order or number of components. Nor should the use of enumeration be construed as excluding from the scope of the claims any additional steps or components that might be combined with or into the enumerated steps or components.

### BRIEF DESCRIPTION OF THE DRAWINGS

FIG. 1 is a perspective view of a thermal control system according to one aspect of the present disclosure shown applied to a patient on a patient support apparatus;

FIG. 2 is a perspective view of a thermal control unit of the thermal control system of FIG. 1;

FIG. 3 is a block diagram of a first embodiment of the thermal control system of FIG. 1;

FIG. 4 is a high-level system diagram including the thermal control system of FIG. 1 according to one aspect of the present disclosure;

FIG. 5 is an exemplary system overview shown on an electronic device according to one aspect of the present disclosure;

FIG. 6 is a high-level system diagram including the thermal control system of FIG. 1 according to one aspect of the present disclosure of the present disclosure;

FIG. 7 is a maintenance screen shown on a user interface of the thermal control system of FIG. 1;

FIGS. 8A-8R are a series of disinfection steps shown on a user interface of the thermal control system of FIG. 1; and

FIGS. 9A-9B are exemplary disinfection overdue messages shown on a user interface of the thermal control system of FIG. 1.

### DETAILED DESCRIPTION OF THE EMBODIMENTS

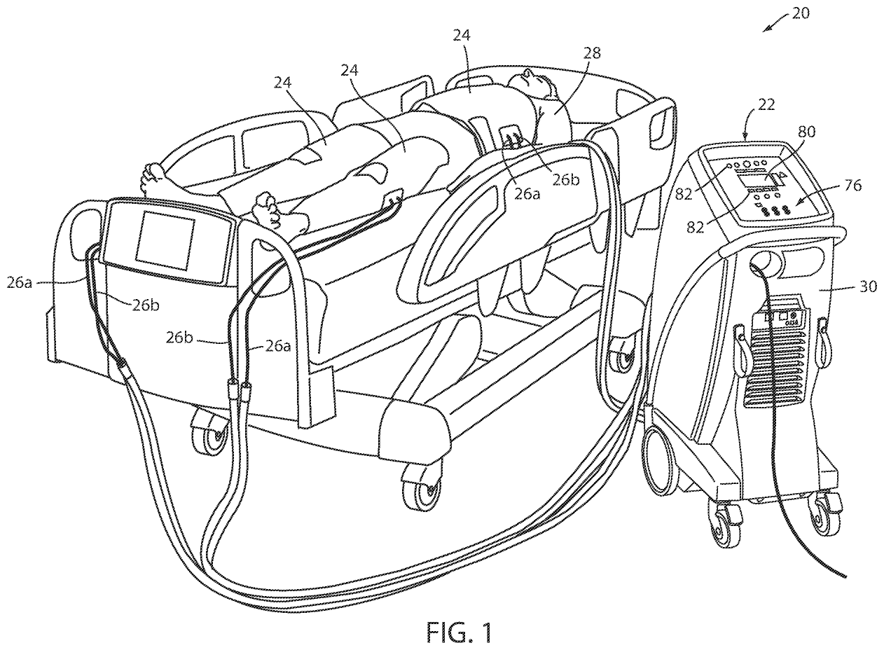

A thermal control system 20 according to one aspect of the present disclosure is shown in FIG. 1. Thermal control system 20 is adapted to control the temperature of a patient 28, which may involve raising, lowering, and/or maintaining the patient's temperature. Thermal control system 20 includes a thermal control unit 22 coupled to one or more thermal therapy devices 24. The thermal therapy devices 24 are illustrated in FIG. 1 to be thermal pads, but it will be understood that thermal therapy devices 24 may take on other forms, such as, but not limited to, blankets, vests, wraps, patches, caps, catheters, or other structures that receive temperature-controlled fluid. For purposes of the following written description, thermal therapy devices 24 will be referred to as thermal pads 24, but it will be understood by those skilled in the art that this terminology is used merely for convenience and that the phrase “thermal pad” is intended to cover all of the different variations of thermal therapy devices 24 mentioned above (e.g. blankets, vests, patches, wraps, caps, catheters, etc.) and variations thereof.

Thermal control unit 22 is coupled to thermal pads 24 via a plurality of hoses 26. Thermal control unit 22 delivers temperature-controlled fluid (such as, but not limited to, water or a water mixture) to the thermal pads 24 via the fluid supply hoses 26a. After the temperature-controlled fluid has passed through thermal pads 24, thermal control unit 22 receives the temperature-controlled fluid back from thermal pads 24 via the return hoses 26b.

In the aspect of thermal control system 20 shown in FIG. 1, three thermal pads 24 are used in the treatment of patient 28. A first thermal pad 24 is wrapped around a patient's torso, while second and third thermal pads 24 are wrapped, respectively, around the patient's right and left legs. Other configurations can be used and different numbers of thermal pads 24 may be used with thermal control unit 22, depending upon the number of inlet and outlet ports that are included with thermal control unit 22. By controlling the temperature of the fluid delivered to thermal pads 24 via supply hoses 26a, the temperature of the patient 28 can be controlled via the close contact of the pads 24 with the patient 28 and the resultant heat transfer therebetween.

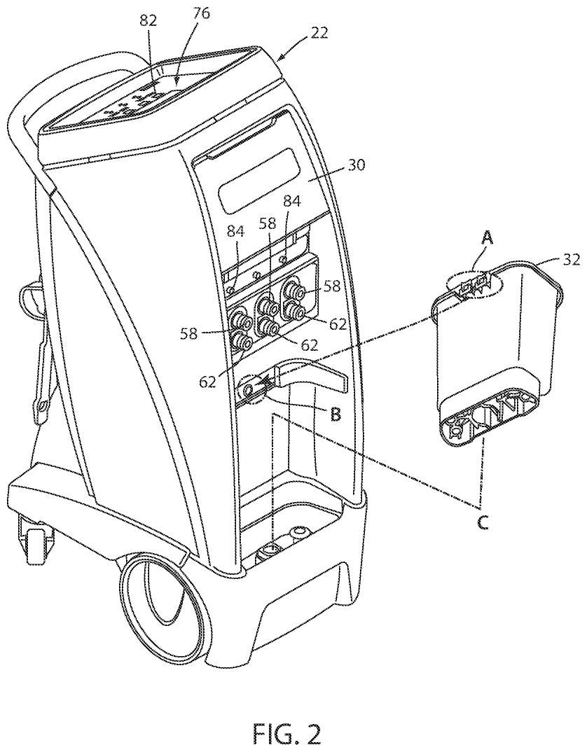

As shown more clearly in FIG. 2, thermal control unit 22 includes a main body 30 to which a removable reservoir 32 may be coupled and uncoupled. Removable reservoir 32 is configured to hold the fluid that is to be circulated through thermal control unit 22 and the one or more thermal pads 24. By being removable from thermal control unit 22, reservoir 32 can be easily carried to a sink or faucet for filling and/or dumping of the water or other fluid. This allows users of thermal control system 20 to more easily fill thermal control unit 22 prior to its use, as well as to drain thermal control unit 22 after use.

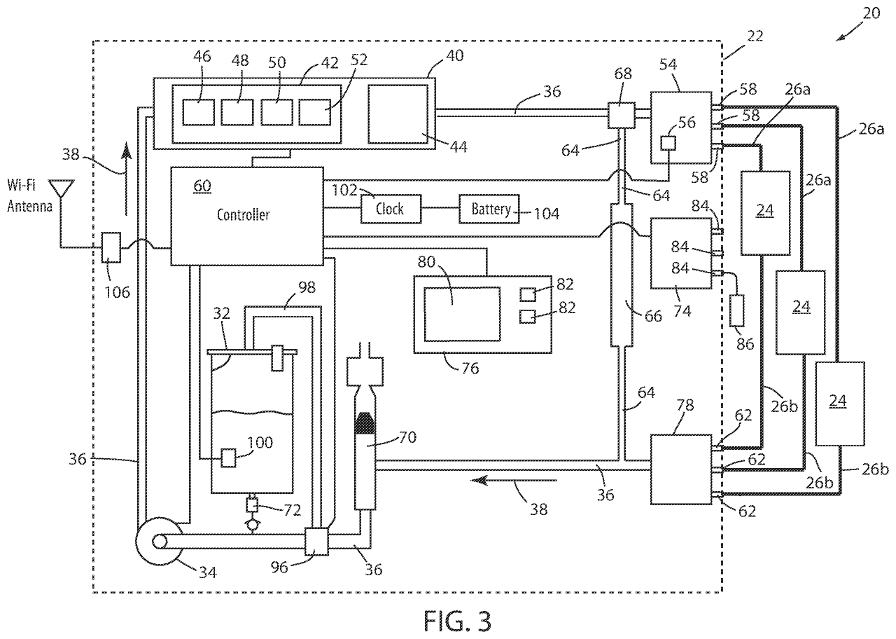

As shown in FIG. 3, thermal control unit 22 includes a pump 34 for circulating fluid through a circulation channel 36. Pump 34, when activated, circulates the fluid through circulation channel 36 in the direction of arrows 38 (clockwise in FIG. 3). Starting at pump 34 the circulating fluid first passes through a heat exchanger 40 that adjusts, as necessary, the temperature of the circulating fluid. Heat exchanger 40 may take on a variety of different forms. In some embodiments, heat exchanger 40 is a thermoelectric heater and cooler. In the embodiment shown in FIG. 3, heat exchanger 40 includes a chiller 42 and a heater 44. Further, in the embodiment shown in FIG. 3, chiller 42 is a conventional vapor-compression refrigeration unit having a compressor 46, a condenser 48, an evaporator 50, an expansion valve (not shown), and a fan 52 for removing heat from the compressor 46. Other types of chillers and/or heaters may be used.

After passing through heat exchanger 40, the circulating fluid is delivered to an outlet manifold 54 having an outlet temperature sensor 56 and a plurality of outlet ports 58. Temperature sensor 56 is adapted to detect a temperature of the fluid inside of outlet manifold 54 and report it to a controller 60. Outlet ports 58 are coupled to supply hoses 26a. Supply hoses 26a are coupled, in turn, to thermal pads 24 and deliver temperature-controlled fluid to the thermal pads 24. The temperature-controlled fluid, after passing through the thermal pads 24, is returned to thermal control unit 22 via return hoses 26b. Return hoses 26b couple to a plurality of inlet ports 62. Inlet ports 62 are fluidly coupled to an inlet manifold 78 inside of thermal control unit 22.

Thermal control unit 22 also includes a bypass line 64 fluidly coupled to outlet manifold 54 and inlet manifold 78 (FIG. 3). Bypass line 64 allows fluid to circulate through circulation channel 36 even in the absence of any thermal pads 24 or hoses 26a being coupled to any of outlet ports 58. In the illustrated embodiment, bypass line 64 includes an optional filter 66 that is adapted to filter the circulating fluid. If included, filter 66 may be a particle filter adapted to filter out particles within the circulating fluid that exceed a size threshold, or filter 66 may be a biological filter adapted to purify or sanitize the circulating fluid, or it may be a combination of both. In some embodiments, filter 66 is constructed and/or positioned within thermal control unit 22 in any of the manners disclosed in commonly assigned U.S. patent application Ser. No. 62/404,676 filed Oct. 11, 2016, by inventors Marko Kostic et al. and entitled THERMAL CONTROL SYSTEM, the complete disclosure of which is incorporated herein by reference.

The flow of fluid through bypass line 64 is controllable by way of a bypass valve 68 positioned at the intersection of bypass line 64 and outlet manifold 54 (FIG. 3). When open, bypass valve 68 allows fluid to flow through circulation channel 36 to outlet manifold 54, and from outlet manifold 54 to the connected thermal pads 24. When closed, bypass valve 68 stops fluid from flowing to outlet manifold 54 (and thermal pads 24) and instead diverts the fluid flow along bypass line 64. In some embodiments, bypass valve 68 may be controllable such that selective portions of the fluid are directed to outlet manifold 54 and along bypass line 64. In some embodiments, bypass valve 68 is controlled in any of the manners discussed in commonly assigned U.S. patent application Ser. No. 62/610,319, filed Dec. 26, 2017, by inventors Gregory Taylor et al. and entitled THERMAL SYSTEM WITH OVERSHOOT REDUCTION, the complete disclosure of which is incorporated herein by reference.

The incoming fluid flowing into inlet manifold 78 from inlet ports 62 and/or bypass line 64 travels back toward pump 34 and into an air remover 70. Air remover 70 includes any structure in which the flow of fluid slows down sufficiently to allow air bubbles contained within the circulating fluid to float upwardly and escape to the ambient surroundings. In some embodiments, air remover 70 is constructed in accordance with any of the configurations disclosed in commonly assigned U.S. patent application Ser. No. 15/646,847 filed Jul. 11, 2017, by inventor Gregory S. Taylor and entitled THERMAL CONTROL SYSTEM, the complete disclosure of which is hereby incorporated herein by reference. After passing through air remover 70, the circulating fluid flows past a valve 72 positioned beneath fluid reservoir 32. Fluid reservoir 32 supplies fluid to thermal control unit 22 and circulation channel 36 via valve 72, which may be a conventional check valve, or other type of valve, that automatically opens when reservoir 32 is coupled to thermal control unit 22 and that automatically closes when reservoir 32 is decoupled from thermal control unit 22 (see FIG. 2). After passing by valve 72, the circulating fluid travels to pump 34 and the circuit is repeated.

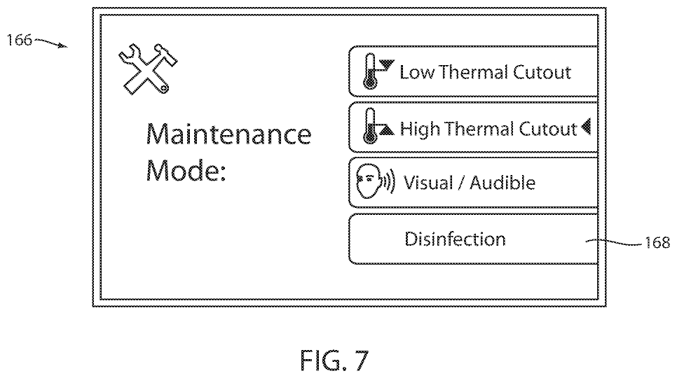

As shown in FIG. 3, the thermal control unit 22 can include a clock 102. In one aspect the clock 102 is a real-time clock. The clock 102 may keep track of the current date and time. The thermal control unit 22 can use the clock 102 to determine whether an internal disinfection cycle is due. This may also be referred to as a disinfection due date. Throughout the disclosure, the terms disinfection cycle and disinfection process are used interchangeably. The thermal control unit 22 may record a disinfection completion time (meaning the time a disinfection cycle completes) using the clock 102 and a disinfection control (described in more detail below with reference to FIG. 7.) The thermal control unit 22 can use a disinfection schedule and the disinfection completion time to determine when the next disinfection process is due. For example, the disinfection schedule may indicate that the thermal control unit 22 should be internally disinfected every 14 days. The clock 102 may be powered by the power supply of the thermal control unit 22 when the thermal control unit 22 is powered. When the thermal control unit 22 is not powered, the clock 102 can be powered by a battery 104. The battery 104 may be any suitable power source. In one aspect, the battery 104 may be a rechargeable battery connected to the power supply of the thermal control unit 22. When the thermal control unit 22 is being powered, the power supply can recharge the battery 104. The thermal control unit 22 can alert the user that disinfection is due using a user interface 76.

In one aspect, the disinfection schedule may require disinfection of the thermal control unit 22 14 days after first use. The thermal control unit 22 can use the clock 102 to record a time of first use. The thermal control unit 22 may then alert the user that a disinfection process is due 14 days after the time of first use.

Controller 60 of thermal control unit 22 is contained within main body 30 of thermal control unit 22 and is in electrical communication with pump 34, heat exchanger 40, outlet temperature sensor 56, bypass valve 68, a patient temperature module 74, and a user interface 76. Controller 60 includes any and all electrical circuitry and components necessary to carry out the functions and algorithms described herein, as would be known to one of ordinary skill in the art. Generally speaking, controller 60 may include one or more microcontrollers, microprocessors, and/or other programmable electronics that are programmed to carry out the functions described herein. It will be understood that controller 60 may also include other electronic components that are programmed to carry out the functions described herein, or that support the microcontrollers, microprocessors, and/or other electronics. The other electronic components include, but are not limited to, one or more field programmable gate arrays, systems on a chip, volatile or nonvolatile memory, discrete circuitry, integrated circuits, application specific integrated circuits (ASICs) and/or other hardware, software, or firmware, as would be known to one of ordinary skill in the art. Such components can be physically configured in any suitable manner, such as by mounting them to one or more circuit boards, or arranging them in other manners, whether combined into a single unit or distributed across multiple units. Such components may be physically distributed in different positions in thermal control unit 22, or they may reside in a common location within thermal control unit 22. When physically distributed, the components may communicate using any suitable serial or parallel communication protocol, such as, but not limited to, CAN, LIN, Firewire, I-squared-C, RS-232, RS-465, universal serial bus (USB), etc.

User interface 76, which may be implemented as a control panel or in other manners, allows a user to operate thermal control unit 22. User interface 76 communicates with controller 60 and includes a display 80 and a plurality of dedicated controls 82. Display 80 may be implemented as a touch screen, or, in some embodiments, as a non-touch-sensitive display. Dedicated controls 82 may be implemented as buttons, switches, dials, or other dedicated structures. In any of the embodiments, one or more of the functions carried out by a dedicated control 82 may be replaced or supplemented with a touch screen control that is activated when touched by a user. Alternatively, in any of the embodiments, one or more of the controls that are carried out via a touch screen can be replaced or supplemented with a dedicated control 82 that carries out the same function when activated by a user.

Through either dedicated controls 82 and/or a touch screen display (e.g. display 80), user interface 76 enables a user to turn thermal control unit 22 on and off, select a mode of operation, select a target temperature for the fluid delivered to thermal pads 24, select a patient target temperature, and control other aspects of thermal control unit 22. In some embodiments, user interface 76 may include a pause/event control, a medication control, and/or an automatic temperature adjustment control that operate in accordance with the pause event control, medication control, and automatic temperature adjustment control disclosed in commonly assigned U.S. patent application Ser. No. 62/577,772 filed on Oct. 27, 2017, by inventors Gregory Taylor et al. and entitled THERMAL SYSTEM WITH MEDICATION INTERACTION, the complete disclosure of which is incorporated herein by reference. Such controls may be activated as touch screen controls or dedicated controls 82.

In those aspects where user interface 76 allows a user to select from different modes for controlling the patient's temperature, the different modes include, but are not limited to, a manual mode and an automatic mode, both of which may be used for cooling and heating the patient. In the manual mode, a user selects a target temperature for the fluid that circulates within thermal control unit 22 and that is delivered to thermal pads 24. Thermal control unit 22 then makes adjustments to heat exchanger 40 in order to ensure that the temperature of the fluid exiting supply hoses 26a is at the user-selected temperature.

When the user selects the automatic mode, the user selects a target patient temperature, rather than a target fluid temperature. After selecting the target patient temperature, controller 60 makes automatic adjustments to the temperature of the fluid in order to bring the patient's temperature to the desired patient target temperature. In this mode, the temperature of the circulating fluid may vary as necessary in order to bring about the target patient temperature.

In order to carry out the automatic mode, thermal control unit 22 utilizes patient temperature module 74. Patient temperature module 74 includes one or more patient temperature sensor ports 84 (FIGS. 2 & 3) that are adapted to receive one or more conventional patient temperature sensors or probes 86. The patient temperature sensors 86 may be any suitable patient temperature sensor that is able to sense the temperature of the patient at the location of the sensor. In one embodiment, the patient temperature sensors are conventional Y.S.I. 400 probes marketed by YSI Incorporated of Yellow Springs, Ohio, or probes that are YSI 400 compliant. In other embodiments, different types of sensors may be used with thermal control unit 22. Regardless of the specific type of patient temperature sensor used in thermal control system 20, each temperature sensor 86 is connected to a patient temperature sensor port 84 positioned on thermal control unit 22. Patient temperature sensor ports 84 are in electrical communication with controller 60 and provide current temperature readings of the patient's temperature. Patient temperature sensor 86 may be positioned in the patient's esophagus, rectum, or other location where temperature readings are indicative of the core temperature of the patient.

Controller 60, in some embodiments, controls the temperature of the circulating fluid using closed-loop feedback from temperature sensor 56. That is, controller 60 determines (or receives) a target temperature of the fluid, compares it to the measured temperature from sensor 56, and issues a command to heat exchanger 40 that seeks to decrease the difference between the desired fluid temperature and the measured fluid temperature. In some embodiments, the difference between the fluid target temperature and the measured fluid temperature is used as an error value that is input into a conventional Proportional, Integral, Derivative (PID) control loop. That is, controller 60 multiplies the fluid temperature error by a proportional constant, determines the derivative of the fluid temperature error over time and multiplies it by a derivative constant, and determines the integral of the fluid temperature error over time and multiplies it by an integral constant. The results of each product are summed together and converted to a heating/cooling command that is fed to heat exchanger 40 and tells heat exchanger 40 whether to heat and/or cool the circulating fluid and how much heating/cooling power to use.

When thermal control unit 22 is operating in the automatic mode, controller 60 may use a second closed-loop control loop that determines the difference between a patient target temperature 88 and a measured patient temperature. The patient target temperature is input by a user of thermal control unit 22 using user interface 76. Measured patient temperature comes from a patient temperature sensor 86 coupled to one of patient temperature sensor ports 84 (FIG. 3). Controller 60 determines the difference between the patient target temperature 88 and the measured patient temperature and, in some embodiments, uses the resulting patient temperature error value as an input into a conventional PID control loop. As part of the PID loop, controller 60 multiplies the patient temperature error by a proportional constant, multiplies a derivative of the patient temperature error over time by a derivative constant, and multiplies an integral of the patient temperature error over time by an integral constant. The three products are summed together and converted to a target fluid temperature value. The target fluid temperature value is then fed to the first control loop discussed above, which uses it to compute a fluid temperature error.

It will be understood by those skilled in the art that other types of control loops may be used. For example, controller 60 may utilize one or more PI loops, PD loops, and/or other types of control equations. In some embodiments, the coefficients used with the control loops may be varied by controller 60 depending upon the patient's temperature reaction to the thermal therapy, among other factors. One example of such dynamic control loop coefficients is disclosed in commonly assigned U.S. patent application Ser. No. 62/577,772 filed on Oct. 27, 2017, by inventors Gregory Taylor et al. and entitled THERMAL SYSTEM WITH MEDICATION INTERACTION, the complete disclosure of which is incorporated herein by reference.

Regardless of the specific control loop utilized, controller 60 implements the loop(s) multiple times a second in at least one embodiment, although it will be understood that this rate may be varied widely. After controller 60 has output a heat/cool command to heat exchanger 40, controller 60 takes another patient temperature reading (from sensor 86) and/or another fluid temperature reading (from sensor 56) and re-performs the loop(s). The specific loop(s) used, as noted previously, depends upon whether thermal control unit 22 is operating in the manual mode or automatic mode.

It will also be understood by those skilled in the art that the output of any control loop used by thermal control unit 22 may be limited such that the temperature of the fluid delivered to thermal pads 24 never strays outside of a predefined maximum and a predefined minimum. Minimum temperature is designed as a safety temperature may be set to about four degrees Celsius, although other temperatures may be selected. The predefined maximum temperature is also implemented as a safety measure and may be set to about forty degrees Celsius, although other values may be selected.

In the embodiment shown in FIG. 3, thermal control unit 22 also includes a reservoir valve 96 that is adapted to selectively move fluid reservoir 32 into and out of line with circulation channel 36. Reservoir valve 96 is positioned in circulation channel 36 between air remover 70 and valve 72, although it will be understood that reservoir valve 96 may be moved to different locations within circulation channel 36. Reservoir valve 96 is coupled to circulation channel 36 as well as a reservoir channel 98. When reservoir valve 96 is open, fluid from air remover 70 flows along circulation channel 36 to pump 34 without passing through reservoir 32 and without any fluid flowing along reservoir channel 98. When reservoir valve 96 is closed, fluid coming from air remover 70 flows along reservoir channel 98, which feeds the fluid into reservoir 32. Fluid inside of reservoir 32 then flows back into circulation channel 36 via valve 72. Once back in circulation channel 36, the fluid flows to pump 34 and is pumped to the rest of circulation channel 36 and thermal pads 24 and/or bypass line 64. In some embodiments, reservoir valve 96 is either fully open or fully closed, while in other embodiments, reservoir valve 96 may be partially open or partially closed. In either case, reservoir valve 96 is under the control of controller 60.

Thermal control unit 22 also includes a reservoir temperature sensor 100 (FIG. 3). Reservoir temperature sensor 100 reports its temperature readings to controller 60. When reservoir valve 96 is open, the fluid inside of reservoir 32 stays inside of reservoir 32 (after the initial drainage of the amount of fluid needed to fill circulation channel 36 and thermal pads 24). This residual fluid is substantially not affected by the temperature changes made to the fluid within circulation channel 36 as long as reservoir valve 96 remains open. This is because the residual fluid that remains inside of reservoir 32 after circulation channel 36 and thermal pads 24 have been filled does not pass through heat exchanger 40 and remains substantially thermally isolated from the circulating fluid. Two results flow from this: first, heat exchanger 40 does not need to expend energy on changing the temperature of the residual fluid in reservoir 32, and second, the temperature of the circulating fluid in circulation channel 36 will deviate from the temperature of the residual fluid as the circulating fluid circulates through heat exchanger 40.

In some embodiments, controller 60 utilizes a temperature control algorithm to control reservoir valve 96 that is the same as the temperature control algorithm 160 disclosed in commonly assigned U.S. patent application Ser. No. 62/577,772 filed on Oct. 27, 2017, by inventors Gregory Taylor et al. and entitled THERMAL SYSTEM WITH MEDICATION INTERACTION, the complete disclosure of which is incorporated herein by reference. In other embodiments, controller 60 utilizes a different control algorithm. In still other embodiments, thermal control unit 22 is modified to omit reservoir valve 96, reservoir channel 98, and reservoir temperature sensor 100. Thermal control unit 22 may also be modified such that reservoir 32 is always in the path of circulation channel 36. Still other modifications are possible.

It will be understood by those skilled in the art that the thermal control unit 22 can be modified in many different ways. Some possible alternatives are outlined in commonly assigned U.S. patent application Ser. number 2019/0192339 filed Dec. 26, 2017, by inventors Gregory S. Taylor et al. and entitled THERMAL SYSTEM WITH GRAPHICAL USER INTERFACE, the complete disclosure of which is incorporated herein by reference.

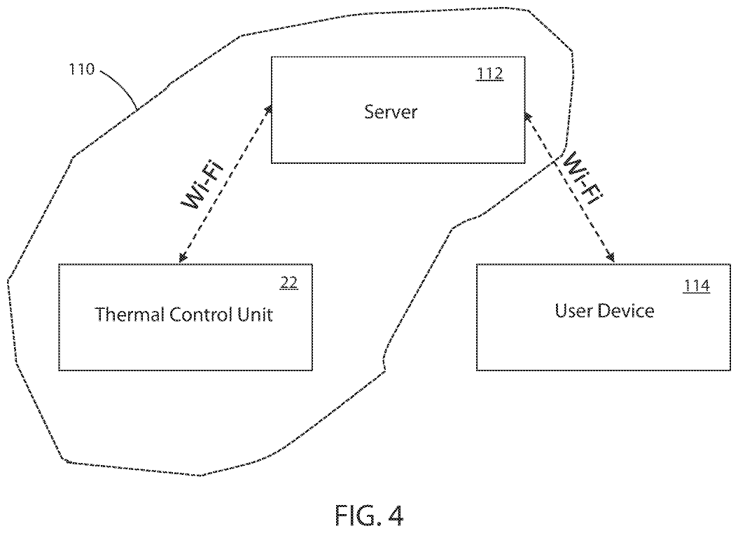

Returning to FIG. 3, thermal control unit 22 includes a communication interface 106 which, in some embodiments, is a wireless transceiver, such as, but not limited to, a Wi-Fi transceiver. In other embodiments, the Wi-Fi transceiver 106 may be replaced with a transceiver that uses a different communication protocol. For example, the thermal control unit 22 may include a cellular data transceiver in place of the Wi-Fi transceiver 106. Turning to FIG. 4, the thermal control unit 22 can be part of a disinfection reminder system 110. The disinfection reminder system 110 may include a server 112 that is adapted to communicate with one or more electronic devices 114. In one aspect, the server 112 is a cloud-based software application. In other aspects, server 112 is a local software application that runs, either partially or wholly, on a physical server positioned inside of the healthcare facility in which thermal control unit 22 is positioned. In either case, thermal control unit 22 uses its Wi-Fi transceiver 106 to communicate with the healthcare facility's local area network (via one or more conventional wireless access points), and the server 112 is either hosted on the healthcare facility's local area network, or accessible from the healthcare facility's local area network through its connection to the Internet.

Electronic devices 114 may be any suitable electronic devices capable of communicating with the server 112. For example, in one aspect, electronic devices 114 may be a cellular phone, a laptop computer, a tablet, a desktop computer, or a combination of any of these. The electronic devices 114 communicate with server 112 communicates through their connection to the healthcare facility's local area network.

It will be noted by one of skill in the art that other configurations of the disinfection reminder system 110 are possible. For example, in one aspect, the thermal control unit 22 may communicate directly with the electronic device 114. In another aspect, the disinfection reminder system 110 can include additional connected devices and communication links.

When the thermal control unit 22 is powered, controller 60 knows the current date and time through the clock 102 and can notify the user of the disinfection status of the thermal control unit 22. For example, the thermal control unit 22 can use the user interface 76 to notify a user that internal disinfection is due in two days' time or is five days overdue. In addition to being displayed locally on display 80, this disinfection status may also be sent to server 112, which can then send a disinfection notification to the organization responsible for the thermal control unit 22 via the electronic device 114. In one aspect, the disinfection notification may be in the form of an email or a short message service (“SMS”) message. The server 112 may send the disinfection notification to the electronic device 114 before the thermal control unit 22 is due for disinfection. For example, the server 112 can send a disinfection notification to the electronic device 114 that a particular thermal control unit 22 will be due for disinfection in three days.

The thermal control unit 22 can communicate the time the last disinfection cycle was completed (also known as the disinfection completion time), the disinfection schedule, and/or the times the thermal control unit 22 is being used to the server 112. The server can store the information received from the thermal control unit 22 and can forward the information to the electronic device 114. In an alternate aspect, the server 112 may select certain pieces of information to forward to the electronic device 114. In one aspect, the server 112 may send information to the electronic device 114 periodically. In another aspect, the server 112 can send information to the electronic device 114 when the electronic device 114 requests information. In another aspect, the server 112 can send information to the electronic device 114 when the server 112 receives information from the thermal control unit 22. The server 112 has the ability to send the disinfection notification 24 hours a day whereas the thermal control unit 22 can only alert a user when it is powered on.

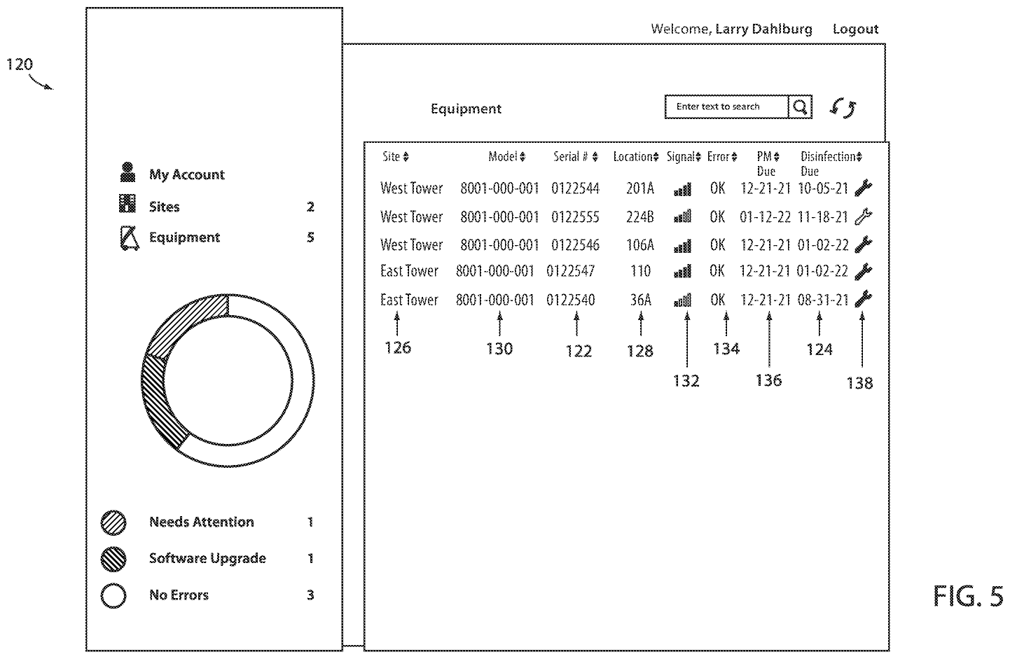

Based on the information received from the thermal control unit 22, the server 112 can transmit an internal disinfection status of the thermal control unit 22 to the electronic device 114. The internal disinfection status can indicate to the electronic device 114 whether the thermal control unit 22 requires disinfection and/or where the thermal control unit 22 is in its disinfection cycle. The disinfection cycle includes the disinfection process and the time between disinfection processes. The internal disinfection status may be at least one of: up to date, disinfection required soon, disinfection required, and disinfection overdue. The thresholds at which the internal disinfection status changes from one status to another may vary based on the application and may be configurable. For example, in one aspect, the server 112 may send an internal disinfection status of disinfection required soon when the thermal control unit 22 is due for disinfection within five days. In one aspect, the internal disinfection status may include a numeric indicator of how many days until the next disinfection process is required. In another aspect, the internal disinfection status can include a numeric indicator of how many days have passed since the most recent internal disinfection process. The electronic device 114 can display a disinfection indicator corresponding to the internal disinfection status. For example, the electronic device 114 may display a disinfection due date 124 as shown and described below with reference to FIG. 5. In one aspect, the server 112 may send a disinfection due date to the electronic device 114 based on the disinfection completion time and the disinfection schedule and the electronic device 114 may display the disinfection due date.

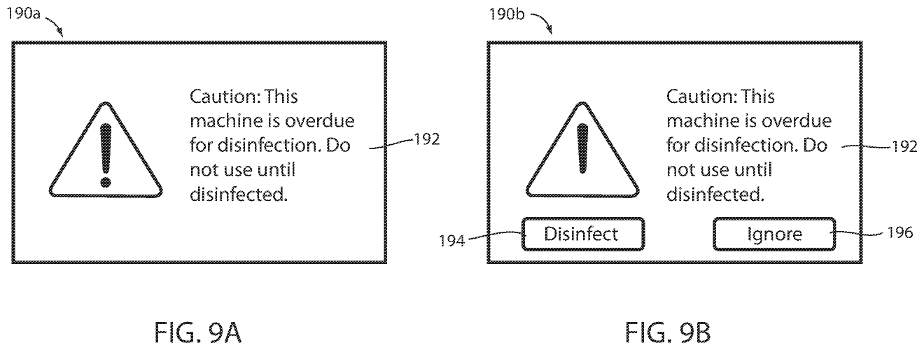

In one aspect, the electronic device 114 may display a different icon for each of the different internal disinfection statuses. For example, the electronic device 114 may display a check mark when the internal disinfection status is up to date, a warning sign when the internal disinfection status is disinfection required soon, a stop sign when the internal disinfection status is disinfection required, and an exclamation mark when the internal disinfection status is disinfection overdue. Additionally, or alternatively, the electronic device 114 may indicate the internal disinfection status using different colors to represent the different internal disinfection statuses. The electronic device 114 can display a disinfection due indicator when the electronic device 114 receives the internal disinfection status of disinfection required or disinfection overdue. In one aspect, the disinfection due indicator may be a pop-up screen similar to the ones shown and described below with reference to FIGS. 9A-9B. The server 112 may send the internal disinfection status to the electronic device 114 at a predetermined interval. In one aspect, the predetermined interval is one day. In one aspect, the electronic device 114 can display a visual indication of whether disinfection is due for each thermal control unit 22. In some embodiments, any of the icons and/or indicators that are displayed on an electronic device 114 regarding a particular thermal control unit 10 may alternatively, or additionally, be displayed on display 80 of the thermal control unit 22.

The thermal control unit 22 can track the internal disinfection status itself using the clock 102 and the disinfection schedule. In one aspect, the thermal control unit 22 may receive the internal disinfection status from the server 112. The thermal control unit 22 may require the user to run the disinfection process when the internal disinfection status is disinfection required or disinfection overdue. In one aspect, the thermal control unit 22 may display the disinfection overdue screen 190 shown and described below with reference to FIG. 9A to require the user to run the disinfection process. The user interface 76 may display a disinfection due message when the internal disinfection status is disinfection required or disinfection overdue. The user may be able to bypass the disinfection due message and operate the thermal control unit 22 as shown and described below with reference to FIG. 9B. In one aspect, the user interface may display a number of days the disinfection process is overdue when the internal disinfection status is disinfection overdue.

In one aspect, the server 112 stores the disinfection schedule. The server 112 can use the disinfection schedule to notify the electronic device 114 when a thermal control unit 22 requires disinfection. In one aspect, the server 112 can notify the thermal control unit 22 when the unit requires disinfection. This may be instead of or in addition to the thermal control unit 22 itself tracking when disinfection is required.

FIG. 5 shows an exemplary status screen 120 that can be displayed on the electronic device 114. The status screen 120 can display thermal control unit information including an identifier 122; a disinfection due date 124; a site 126; a location 128; a model number 130; a signal status 132; an error message 134; a preventable maintenance indicator 136; and a software update indicator 138. The status screen 120 displays a listing of identifiers 122 for the thermal control units 22 in a particular system. In the example shown in FIG. 5, the identifiers 122 are serial numbers corresponding to individual thermal control units 22 and the status screen 120 lists five identifiers 122. Each thermal control unit 22 has the disinfection due date 124 listed on the status screen 120. The electronic device 114 can change the appearance of the disinfection due date 124 depending on the internal disinfection status of the thermal control unit 22. In one aspect, the electronic device 114 may change the appearance of the disinfection due date when the current date is the same as or later than the current due date. Put another way, the electronic device 114 may change the appearance of the disinfection due date when the internal disinfection status is disinfection required or disinfection overdue. In one aspect, the disinfection due date 124 may be shown in a different color is disinfection for the thermal control unit 22 is overdue. In one aspect, the disinfection due date 124 may be shown in a third color when disinfection is due soon but is not currently due. For example, the disinfection due date 124 may be shown in the third color when the disinfection due date is three days from the current date. In one aspect, the controller 60 may be configured to calculate the amount of time between the current date and the disinfection due date using the clock 102, the disinfection schedule, and the disinfection completion time. In one aspect, the status screen 120 may display a disinfection status indicator in place of the disinfection due date 124 or in addition to the disinfection due date 124. The disinfection status indicator can be a pictorial representation of the disinfection status of each thermal control unit 22. In one aspect, the electronic device 114 may be configured to alert the user when disinfection is due. The alert may occur once or may be repeated until the disinfection process is performed. For example, the electronic device 114 may display a pop-up screen indicating disinfection is due for at least one thermal control unit when a user starts up the electronic device 114.

The status screen 120 can also include the site 126 and the location 128 for each thermal control unit 22. The site 126 and the location 128 may assist the technician in locating the thermal control unit 22. The status screen 120 may also include the model number 130 for the thermal control unit 22. The model number 130 can help the technician to identify the particular thermal control unit 22. The technician may also use the identifier 122 to find the correct thermal control unit 22. The status screen 120 can display the signal status 132. The signal status 132 may show the strength of the signal from the Wi-Fi transceiver 106. If the signal status 132 shows as weak or disconnected, the technician may recognize that the electronic device 114 may not be receiving the necessary information from the thermal control unit 22 and the technician may take corrective action. For example, the technician may move the thermal control unit to another location where the signal strength from the Wi-Fi transceiver 106 is stronger. The status screen 120 can include the error message 134. The error message 134 can display any error with the thermal control unit. For example, the error message 134 may indicate that the disinfection process did not complete successfully or that the removable reservoir 32 is not properly installed in the main body 30. The status screen 120 can include the preventative maintenance indicator 136. The preventative maintenance indicator 136 may operate similarly to the disinfection due date 124 described above to indicate to the operator when preventative maintenance is required on the thermal control unit 22. The status screen 120 can include the software update indicator 138. The software update indicator 138 may indicate whether there is a software update available for the thermal control unit 22. As shown in FIG. 5, the software update indicator 138 is a wrench that is a first color when there is no software update and a second color when there is an available software update.

The status screen 120 may display a global status indicator 140. The global status indicator 140 can be a high-level overview of the entire inventory of thermal control units 22. As shown in FIG. 5, the global status indicator 140 is a donut chart representing all the thermal control units 22 in a particular hospital's system. The global status indicator 140 shows how many thermal control units 22 need attention, have an available software upgrade, or have no errors. The technician can look to the detailed listings of the thermal control units 22 using all of the thermal control unit information described above to determine which thermal control units 22 require attention and what kind of attention they require.

It will be noted that status screen 120 may depart from the example shown in FIG. 5 and still be covered by this disclosure. Status screen 120 can have the same information as shown in FIG. 5 but arranged in a different way. Status screen 120 may display different information from that shown in FIG. 5. In some embodiments, status screen 120 is a web page that is generated by server 112 and accessed using a conventional web-browser running on electronic device 114. In other embodiments, status screen 120 may be generated from a specialized native software application that is executed by electronic device and that retrieves the information shown in screen 120 from server 112. In either case, the information shown on status screen 120 is received from server 112 which, in turn, receives most, if not all, of this information, from the individual thermal control units 22 that are positioned within a particular healthcare facility. In some embodiments, thermal control unit 22 itself may be configured to display any or all of the information shown on status screen 120 on its own display 80.

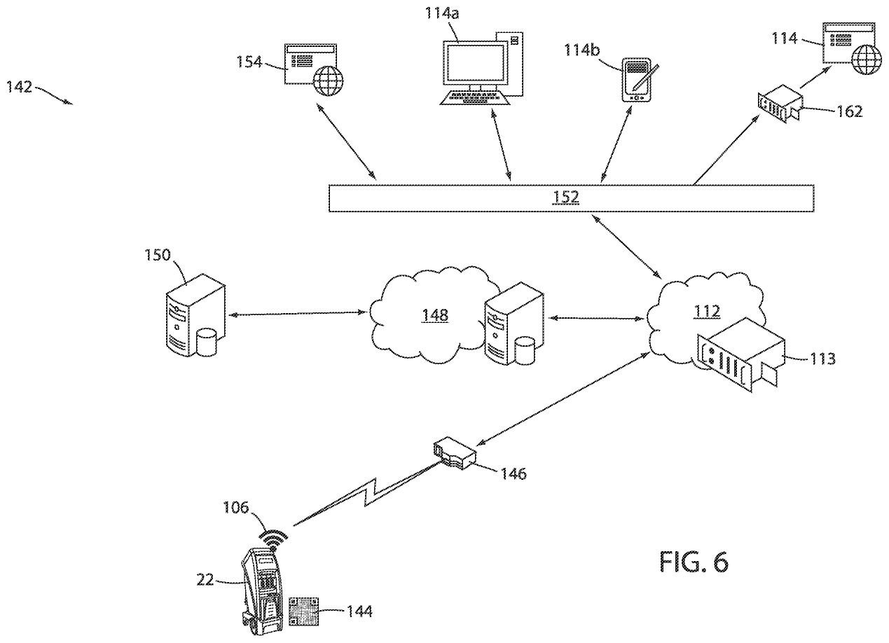

FIG. 6 shows an exemplary network connection configuration 142 for a thermal control unit 22 according to one aspect. All communication links in the network connection configuration 142 can be encrypted as a security measure. Unless otherwise noted, all communication links described below allow for two-way communication. The thermal control unit 22 may generate an identifying code 144 on display 80. The identifying code 144 may alternately be referred to as a therapy session identifier. The identifying code 144 can be a bar code, quick response (“QR”) code, or any other suitable identifier. A technician may scan the identifying code 144 using his or her associated electronic device 114 to obtain information about the thermal control unit 22. For example, the information may include disinfection information, treatment information, or machine maintenance information. The thermal control unit 22 can communicate with an edge gateway 146 using the Wi-Fi transceiver 106. The edge gateway 146 may communicate with the server 112. In one aspect, the server 112 can include a storage element 113. The server 112 may communicate with a second server 148. The second server 148 can provide an interface between the server 112 and a hospital electronic medical record (“EMR”) system 150. In one aspect, the hospital EMR system 150 may be on premises at a hospital. In one aspect, the hospital EMR system 150 can be cloud based.

The server 112 includes an application programming interface (API) 152 that, in the illustrated embodiment, is implemented in a software as a service (“SaaS”) form. Other types of APIs may, of course, be used. API 152 is adapted to connect to a variety of structures, including, but not limited to, one or more electronic devices 114. One such structure is an equipment management system 154. Equipment management system 154 may take on a variety of different forms. In one embodiment, equipment management system may take on any of the forms of the equipment management system 20 disclosed in commonly assigned PCT patent application serial number PCT/US2017/041681 filed Jul. 12, 2017, by inventors David Becker et al. and entitled EQUIPMENT MANAGEMENT SYSTEM, the complete disclosure of which is incorporated herein by reference.

Another structure that API 152 is adapted to connect to is an electronic device 114a that is adapted to execute a conventional web-browser and to access server 112 via the conventional web-browser. In other words, API 152 is configured to allow access to the data of server 112 via a conventional web-page that a user accesses through his or her electronic device 114a (provided they have the proper login credentials). Yet another structure that API 152 is adapted to connect to is an electronic device 114b that executes a native software application that is customized for accessing data from server 112. For example, electronic devices 114b may be a conventional smart phone, tablet, or other type of computer that uses a native software application (which may be downloaded from an existing app store such as Google Play, Apple's App store, etc.) to communicate with server 112 (through API 152). In one aspect, the API 152 may also connect to a bed server 162 that is in communication with one or more beds in the same healthcare facility as thermal control unit 22. The bed server 162 can connect to one or more electronic devices 114.

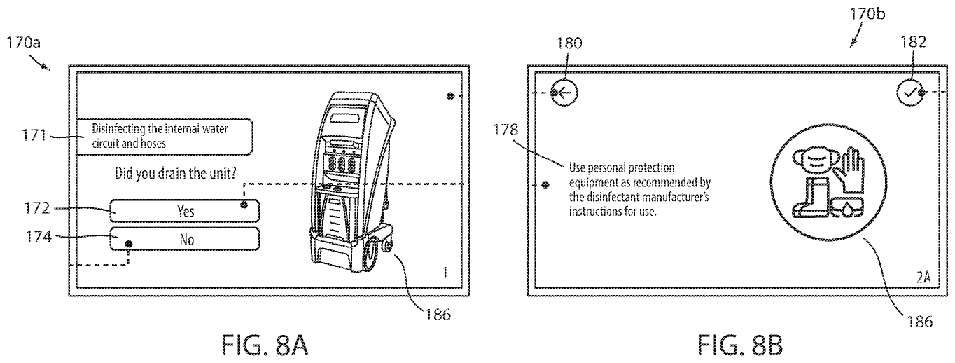

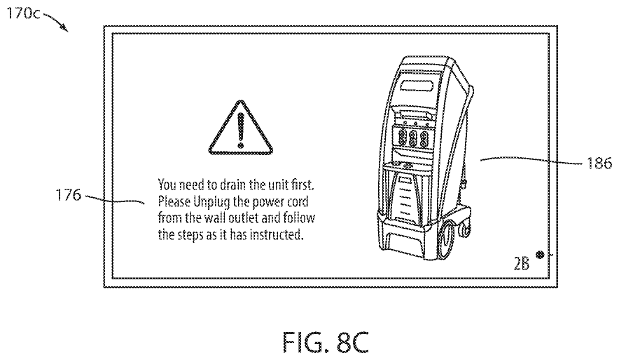

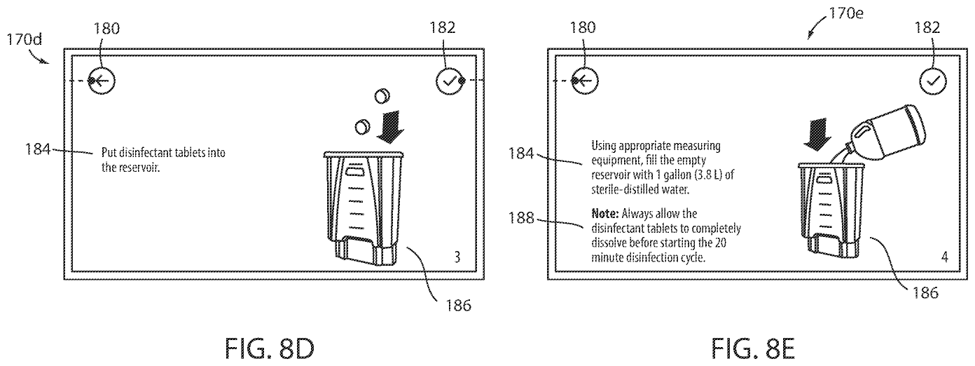

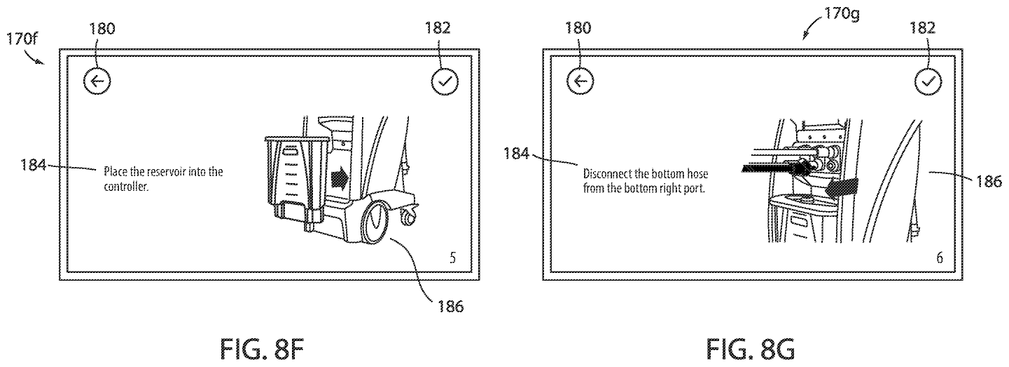

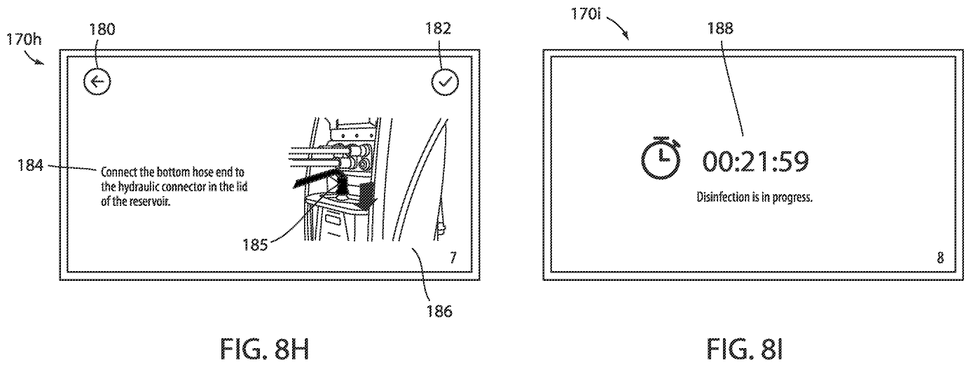

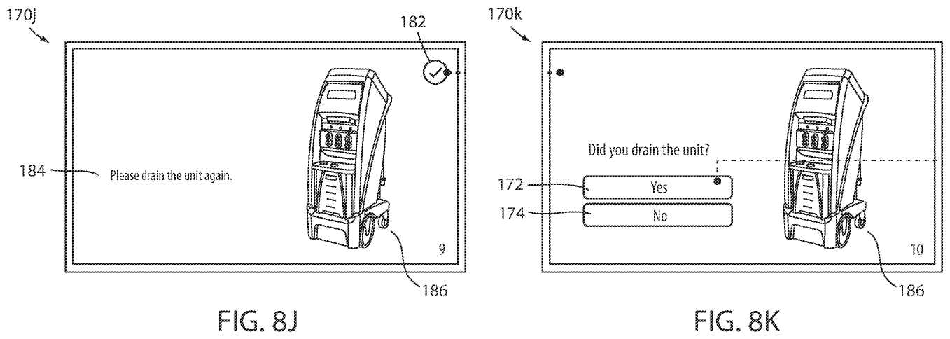

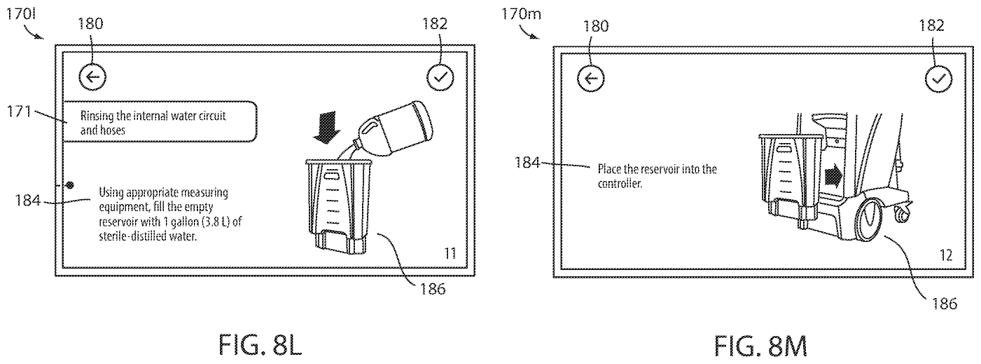

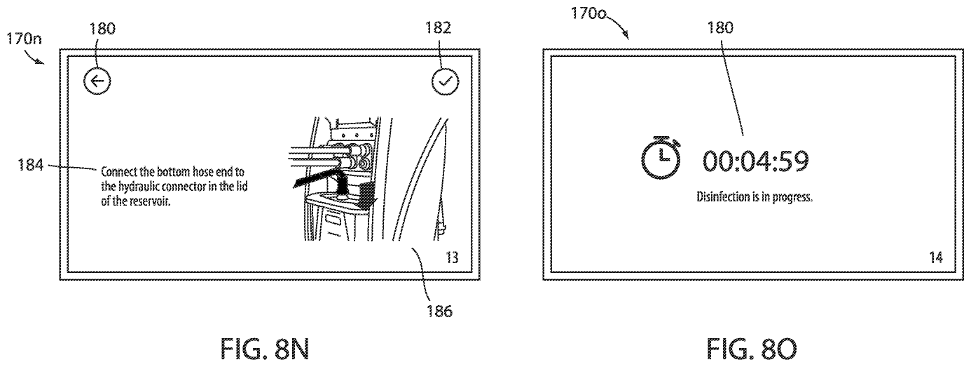

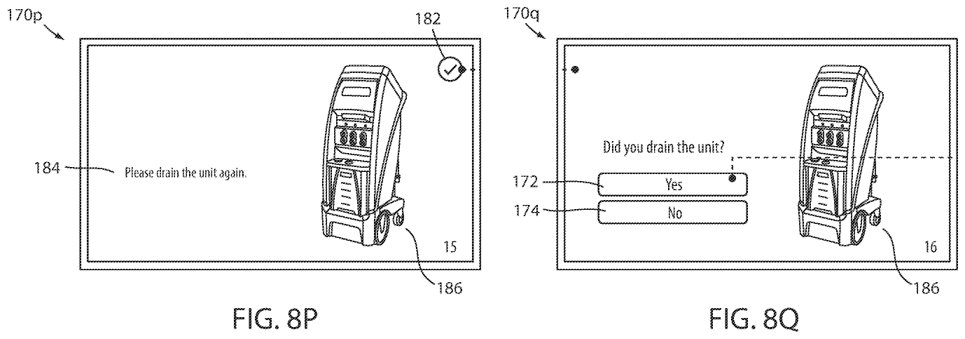

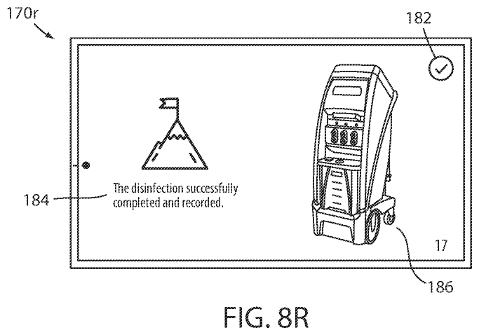

As seen in FIG. 7, user interface 76 includes a disinfection control 168 that triggers a disinfection wizard. The disinfection wizard is a process carried out by controller 60 that guides the user through the disinfection process. Put another way, the disinfection wizard may provide step by step assistance to the user to sequentially walk them through the internal disinfection process. Generally, the disinfection process includes: draining the internal water circuit and hoses for disinfection; disinfecting the internal water circuit and hoses; and rinsing the internal water circuit and hoses. The disinfection wizard breaks down these high-level steps into many more steps for the technician or the thermal control unit 22 to perform. In one aspect, the disinfection process may consist of 61 total steps. The disinfection wizard may instruct the thermal control unit 22 to perform certain actions related to the internal disinfection process. For example, the disinfection wizard can set the water target temperature to a specific value or set a timer for one or more steps in the internal disinfection process. In one aspect, the disinfection wizard may set the target temperature to 25.0 degrees Celsius or 77.0 degrees Fahrenheit. In one aspect, the disinfection wizard can set a 22-minute timer for disinfecting the internal water circuit and hoses. In one aspect, the disinfection wizard can set a five-minute timer for rinsing the internal water circuit and hoses. The disinfection screens 170 that may appear on the user interface 76 to guide a user through the internal disinfection process according to one aspect are shown and described below with reference to FIGS. 8A-8R. The disinfection wizard displays the disinfection screens 170 on the display 80 of the user interface 76 so the technician knows exactly what to do.

The disinfection wizard automates certain steps of the disinfection process. Many of the disinfection steps require at least some interaction by the technician, but some steps can be fully automated. In one aspect, twenty-five percent of the steps may be automated and therefore eliminated from the technician's perspective. This reduces the workload of the technician and simplifies the disinfection process from a user perspective.

FIG. 7 shows an exemplary maintenance screen 166 that appears on the user interface 76 of the thermal control unit 22. As depicted, the maintenance screen 166 includes disinfection control 168. The disinfection control 168 can trigger the disinfection wizard and/or bring up a disinfection screen on the user interface 76 that gives the user information about when the thermal control unit 22 will need disinfection, for example. The disinfection screen can provide a separate control that triggers the disinfection wizard. It will be understood by one of skill in the art that the disinfection control 168 may appear on a different screen on the user interface 76 or may be a separate dedicated control 82.

FIGS. 8A-8R depict an exemplary series of disinfection screens 170 that appear on the user interface 76 to guide a technician through the disinfection process. As depicted in FIGS. 8A-8R, the user interface 76 includes a touch screen and the disinfection screens 170 may be navigated through user input on the touch screen. The disinfection process begins with the disinfection screen 170a depicted in FIG. 8A. The disinfection screen 170a includes a disinfection stage indicator 171. In FIG. 8A, the disinfection stage indicator 171 shows that the disinfection process is in the “disinfecting the internal water circuit and hoses” stage. The first step in the disinfection process is to drain the thermal control unit 22. FIG. 8A includes a diagram 186 of the thermal control unit 22 and asks the technician if they have drained the thermal control unit 22. The technician may either select a yes control 172 if they have drained the unit or a no control 174 if they have not drained the unit. The yes control 172 may alternately be referred to as a yes indicator and the no control 174 may alternately be referred to as a no indicator. If the technician selects the yes control 172, the disinfection wizard moves on to the disinfection screen 170b shown in FIG. 8B. If the technician selects the no control 174, the disinfection wizard proceeds to the disinfection screen 170c shown in FIG. 8C.

FIG. 8C illustrates a disinfection wizard screen 180c that includes a graphical depiction 186 of thermal control unit 22 and a warning 176 that the thermal control unit 22 needs to be drained before the disinfection process can begin. The warning 176 instructs a technician to unplug the power cord from the wall and to follow other directions to drain the unit. When the power cord is plugged back into the wall, the thermal control 22 unit may resume the disinfection wizard. In one aspect, the user interface 76 can be powered by the battery 104 when the thermal control unit 22 is unplugged from the wall. The user interface 76 may then display the instructions for draining the unit rather than having the technician look to an external source. The disinfection wizard may then return to the disinfection screen 170a of FIG. 8A. If the technician selects the no control 174 again, the disinfection wizard will repeat the above steps until the technician confirms the thermal control unit 22 has been drained by selecting the yes control 172.

FIG. 8B illustrates a disinfection screen 170b that controller 60 displays on display 80 as part of the disinfection wizard. Disinfection screen 170b includes a reminder 178 and a diagram 186 that instructs the technician to follow the personal protective equipment (“PPE”) guidelines set by a disinfectant manufacturer when completing the disinfection process. One exemplary disinfectant is the BruClean® TbC™ disinfectant tablet. A back control 180 and a forward control 182 are provided on screen 170. The back control 180 will bring the technician to the previous screen in the disinfection process while the forward control 182 will progress the technician to the next screen in the disinfection process.

FIG. 8D illustrates a disinfection screen 180d that includes a direction 184 and a diagram 186 to instruct the technician on the disinfectant to use in the disinfectant process. In one aspect, the direction 184 may instruct the technician to put two disinfectant tablets into the removable reservoir 32. The diagram 186 is of reservoir 32.

FIG. 8E illustrates a disinfection screen 180e that includes a direction 184 and a diagram 186 to instruct the technician on the amount and type of fluid to use in the disinfection process. In the depicted aspect, the fluid is sterile-distilled water and the amount is one gallon or 3.8 liters. The disinfection screen 170e also includes a note to 188 to remind the technician to allow the disinfectant tablets to fully dissolve before running the disinfection cycle. In an alternate aspect, the disinfection screen 170e can provide a yes control and a no control for the technician to confirm whether the disinfectant tablets are fully dissolved. If the technician selects the no control, the user interface 76 may display a reminder screen to remind the technician to allow the disinfectant tablets to fully dissolve and return to the disinfection screen 170 including the yes control and the no control. If the technician selects the yes control, controller 60 moves to the next step in the disinfection process and displays the disinfection screen 170f shown in FIG. 8F.

Disinfection screen 170f of FIG. 8F includes a direction 184 and a diagram 186 to instruct the technician to place the removable reservoir 32 into the thermal control unit 22.

FIG. 8G illustrates a disinfection screen 170g that shows a direction 184 and a diagram 186 to instruct the technician to disconnect the bottom hose 26 of the thermal control unit 22 from the bottom right port 62.

FIG. 8H illustrates a disinfection screen 170h that includes a direction 184 and a diagram 186 to instruct the technician to connect the hose 26 to a hydraulic connector 185 in a lid of the removable reservoir 32. The hydraulic connector 185 is shown in diagram 186 and provides a connector for hose 26 so that hose 26 may deliver fluid to reservoir 32. By connecting hose 26 to hydraulic connector 185, the fluid circuit of thermal control unit 22 is modified from its normal operation so as to include reservoir 32. In this manner, reservoir 32 is thoroughly disinfected during the disinfection process.

FIG. 81 illustrates a disinfection screen 170i that includes a timer 188 that counts down from a predetermined disinfection time to indicate to the technician when the disinfection step is complete. In the embodiment of FIG. 81, the predetermined disinfection time is 22 minutes. The timer 188 utilizes the clock 102 to accurately count down. During the disinfection process of step 81, controller 60 not only automatically selects the duration for this step (e.g. 22 minutes), but also automatically selects a target temperature for the fluid that is circulating in circulation channel. In some embodiments, the automatically selected target temperature is 25° Celsius, although other target temperatures may be used. Regardless of the particular value that is automatically selected, controller 60 automatically controls heat exchanger 40 so that it changes the temperature of the circulating fluid to the target temperature, and thereafter maintains it at that target temperature for the duration of this step.

FIG. 8J illustrates a disinfection screen 170j that includes a direction 184 and a diagram 186 that instructs the technician to drain the thermal control unit 22 again.

FIG. 8K illustrates a disinfection screen 170k that includes a diagram 186, asks the technician if they have drained the thermal control unit 22, and provides a yes control 172 and a no control 174. If the technician selects the yes control 172, the disinfection wizard proceeds to the disinfection screen 170 shown in FIG. 8L. If the technician selects the no control 174, the disinfection wizard returns to FIG. 8J.

FIG. 8L illustrates a disinfection screen 170I that includes a disinfection stage indicator 171 that tells the technician that the disinfection process is in the “rinsing the internal water circuit and hoses” stage. Disinfection screen 170I also includes a direction 184 to instruct the technician how much and what type of fluid to put in the removable reservoir 32. Additionally, disinfection screen 170I also includes a diagram 186 of the reservoir 32. In the example screen 170I of FIG. 8L, the fluid is sterile-distilled water and the amount is one gallon or 3.8 liters.

FIG. 8M illustrates a disinfection screen 170m that includes a direction 184 and a diagram 186 to instruct the technician to place the removable reservoir 32 into the thermal control unit 22.

FIG. 8N illustrates a disinfection screen 170n that includes a direction 184 and a diagram 186 to instruct the technician to connect the hose 26 from the bottom right port 62 of the thermal control unit 22 to a hydraulic connector in a lid of the removable reservoir 32. Put another way, FIGS. 8M-8N instruct the technician to physically manipulate the thermal control unit 22.

FIG. 8O illustrates a disinfection screen 1700 that includes a timer 188 that counts down from a predetermined disinfection time to indicate to the technician when the disinfection step is complete. In the embodiment of FIG. 8O, the predetermined disinfection time is five minutes. The timer 188 utilizes the clock 102 to accurately count down.

FIG. 8P illustrates a disinfection screen 170p that includes a direction 184 and a diagram 186 to instruct the technician to drain the thermal control unit 22.

FIG. 8Q illustrates a disinfection screen 170q that includes a diagram 186 and asks the technician to drain the unit again and includes a yes control 172 and a no control 174. If the technician selects the yes control 172, the disinfection wizard proceeds to FIG. 8R. If the technician selects the no control 174, the disinfection wizard returns to FIG. 8P.

FIG. 8R illustrates a disinfection screen 170r that includes a direction 184 and a diagram 186 to instruct the technician that the disinfection process is complete. The thermal control unit 22 may record the disinfection completion time by using the clock 102. The forward control 182 ends the disinfection wizard.

Each of the disinfection screens 170A-R may be equipped with a forward control 182 to allow the technician to progress through the disinfection process and/or a back control 180 to return to the previous step in the disinfection process. In an alternate aspect, the disinfection screens 170A-R may be on a timer and may progress when the timer expires unless a user input is required. It will also be understood that the disinfection wizard executed by controller 60 may utilize additional and/or different screens from the disinfection screen 170A-R shown in the accompanying drawings. The content of screens 170A-R may also be modified from what is shown in the attached drawings.

FIGS. 9A and 9B are exemplary disinfection overdue screens 190 that may appear on the user interface 76. The disinfection overdue screen 190 alerts the technician that the thermal control unit 22 is overdue for disinfection. In FIG. 9A, a disinfection overdue screen 190a displays a warning 192 to indicate to the technician that the thermal control unit 22 is overdue for disinfection and should not be used until it has been disinfected. In one aspect, the disinfection overdue screen 190a may remain on the user interface 76 until the disinfection wizard is triggered. In another aspect, the disinfection overdue screen 190a may disappear when the technician causes another item to be displayed on the user interface 76. In yet another aspect, the disinfection overdue screen can remain on the user interface 76 for a predetermined period of time after the thermal control unit 22 is turned on and then disappear from the user interface 76.

In FIG. 9B, a disinfection overdue screen 190b displays a warning 192 to indicate to the technician that the thermal control unit 22 is overdue for disinfection and should not be used until it has been disinfected. The disinfection overdue screen 190b may include a disinfect control 194 and an ignore control 196. When the technician selects the disinfect control 194, the thermal control unit 22 may trigger the disinfection wizard. When the technician selects the ignore control 196, the disinfection overdue screen 190 can disappear from the user interface 76. The disinfection overdue screen 190 may reappear on the user interface 76 periodically until the thermal control unit 22 has been disinfected. In one aspect, the disinfection overdue screen 190b may only include the disinfect control 194 and not the ignore control 196 after either the ignore control 196 has been selected a predefined number of times or if a predetermined amount of time has passed since the first time the disinfection overdue screen 190b appeared on the user interface 76 during this disinfection cycle.

It will also be understood that any of the thermal control units disclosed herein may be modified to additionally operate in conjunction with one or more auxiliary sensors used to sense one or more non-temperature patient parameters. When so modified, any of the thermal control units disclosed herein may utilize the auxiliary sensors in any of the manners, and using any of the structures and/or algorithms, disclosed in commonly assigned U.S. patent application Ser. No. 62/610,327 filed Dec. 26, 2017, by inventors Gregory S. Taylor et al. and entitled THERMAL SYSTEM WITH PATIENT SENSOR(S), the complete disclosure of which is incorporated herein by reference.

Any of the thermal control units disclosed herein may also or alternatively be modified to incorporate any of the temperature overshoot reduction methods, structures, and/or algorithms disclosed in commonly assigned U.S. patent application Ser. No. 62/610,319 filed Dec. 26, 2017, by inventors Gregory Taylor et al. and entitled THERMAL SYSTEM WITH OVERSHOOT REDUCTION, the complete disclosure of which is incorporated herein by reference. Additionally or alternatively, any of the thermal control units disclosed herein may use any of the data and algorithms disclosed in U.S. patent application Ser. No. 62/610,334 filed Dec. 26, 2017, by inventors Christopher Hopper et al. and entitled THERMAL CONTROL SYSTEM when determining when a patient's core temperature will reach its temperature, and/or when to transition from heating the circulating fluid to cooling the circulating fluid, and vice versa, in order to reduce overshoot. The '334 application is hereby incorporated herein by reference in its entirety.

Various other alterations and changes beyond those already mentioned herein can be made to the above-described embodiments. This disclosure is presented for illustrative purposes and should not be interpreted as an exhaustive description of all embodiments or to limit the scope of the claims to the specific elements illustrated or described in connection with these embodiments. For example, and without limitation, any individual element(s) of the described embodiments may be replaced by alternative elements that provide substantially similar functionality or otherwise provide adequate operation. This includes, for example, presently known alternative elements, such as those that might be currently known to one skilled in the art, and alternative elements that may be developed in the future, such as those that one skilled in the art might, upon development, recognize as an alternative. Any reference to claim elements in the singular, for example, using the articles “a,” “an,” “the” or “said,” is not to be construed as limiting the element to the singular.

1. A thermal control unit for controlling a patient's temperature during a thermal therapy session, the thermal control unit comprising:
a fluid outlet adapted to couple to a first hose in fluid communication with a thermal pad in contact with a patient;
a fluid inlet adapted to couple to a second hose in fluid communication with the thermal pad;
a circulation channel coupled to the fluid inlet and the fluid outlet;
a pump for circulating the fluid through the circulation channel from the fluid inlet to the fluid outlet;
a heat exchanger adapted to add or remove heat from the fluid circulating in the circulation channel;
a display;
a disinfection control for initiating a disinfection cycle; and
a controller adapted to control the heat exchanger in order to control the patient's temperature, the controller further adapted to display a series of disinfection steps corresponding to the disinfection cycle in response to the disinfection control being activated, the series of disinfection steps including a particular step in which the controller automatically performs at least one of the following: (a) selects a target temperature for the circulating fluid and controls the heat exchanger such that a temperature of the fluid in the circulation channel is changed to the target temperature; or (b) measures a set amount of time and provides an indication to the user after the set amount of time has elapsed.

2. The thermal control unit of claim 1 wherein the display is part of a touch screen, and the touch screen is adapted to progress through the series of disinfection steps based on user input on the touch screen.

3. The thermal control unit of claim 1 wherein the series of disinfection steps includes at least two of the following:
a step in which the controller displays a message on the display reminding the user to drain the thermal control unit;
a step in which the controller displays a graphical representation of the thermal control unit;
a step in which the controller displays on the display a graphical representation of a piece of equipment used in the disinfection cycle;
a step in which the controller displays a timer counting down from a predetermined time;
a step in which the controller displays instructions to the user to physically manipulate the thermal control unit;
a step in which the controller displays instructions to the user to place a reservoir inside the thermal control unit;
a step in which the controller displays instructions to the user to disconnect a hose from a port of the thermal control unit; or
a step in which the controller displays instructions to the user to connect a hose to a hydraulic connector in a lid of a reservoir.

4. (canceled)

5. The thermal control unit of claim 1 wherein the controller is adapted to display a completion message on the display when the disinfection cycle is completed.

6-12. (canceled)

13. The thermal control unit of claim 3 wherein the series of disinfection steps further includes a step in which the controller is adapted to display instructions to the user to confirm whether the thermal control unit has been drained.

14. The thermal control unit of claim 13 wherein the controller is adapted display a yes indicator and a no indicator and to proceed to a next disinfection step in the series of disinfection steps if the user selects the yes indicator, and to not proceed to the next step if the user selects the no indicator.

15. The thermal control unit of claim 1 further comprising a communication interface adapted to transmit a message to a server when the disinfection cycle has been completed.

16. The thermal control unit of claim 15 wherein the message indicates a time when the disinfection cycle was completed.

17. The thermal control unit of claim 1 wherein the controller is adapted to display an indicator on the display, the indicator indicating when a next disinfection cycle should be performed on the thermal control unit.

18. A thermal control unit for controlling a patient's temperature during a thermal therapy session, the thermal control unit comprising:
a fluid outlet adapted to couple to a first hose in fluid communication with a thermal pad in contact with a patient;
a fluid inlet adapted to couple to a second hose in fluid communication with the thermal pad;
a circulation channel coupled to the fluid inlet and the fluid outlet;
a pump for circulating the fluid through the circulation channel from the fluid inlet to the fluid outlet;
a heat exchanger adapted to add or remove heat from the fluid circulating in the circulation channel;
a display; and
a controller adapted to control the heat exchanger in order to control the patient's temperature, the controller further adapted to display an indicator on the display, the indicator indicating information about a disinfection status of the thermal control unit.

19. The thermal control unit of claim 18 wherein the indicator indicates when a next disinfection cycle should be performed on the thermal control unit.

20. The thermal control unit of claim 18 wherein the indicator indicates a number of days until the next disinfection cycle should be performed.

21. The thermal control unit of claim 18 wherein the indicator indicates at least one of: up to date, disinfection required soon, disinfection required, or disinfection overdue.

22. The thermal control unit of claim 18 further comprising a disinfection control and wherein the controller is further adapted to display a series of disinfection steps for a disinfection cycle in response to the disinfection control being activated, the series of disinfection steps including a particular step in which the controller automatically performs at least one of the following: (a) selects a target temperature for the circulating fluid and controls the heat exchanger such that a temperature of the fluid in the circulation channel is changed to the target temperature; or (b) measures a set amount of time and provides an indication to the user after the set amount of time has elapsed.

23. The thermal control unit of claim 22 wherein the display is part of a touch screen, and the touch screen is adapted to progress through the series of disinfection steps based on user input on the touch screen.

24. The thermal control unit of claim 22 wherein the series of disinfection steps further includes at two of the following:
a step in which the controller displays a message on the display reminding the user to drain the thermal control unit;
a step in which the controller displays a graphical representation of the thermal control unit;
a step in which the controller displays on the display a graphical representation of a piece of equipment used in the disinfection cycle;
a step in which the controller displays a timer counting down from a predetermined time;
a step in which the controller displays instructions to the user to place a reservoir inside the thermal control unit;
a step in which the controller displays instructions to the user to disconnect a hose from a port of the thermal control unit; or
a step in which the controller displays instructions to the user to connect a hose to a hydraulic connector in a lid of a reservoir.

25. (canceled)

26. The thermal control unit of claim 22 wherein the controller is adapted to display a completion message on the display when the disinfection cycle is completed.

27-31. (canceled)

32. The thermal control unit of claim 22 wherein the series of disinfection steps includes a step in which the controller is adapted to display instructions to the user to confirm whether the thermal control unit has been drained, as well as a yes indicator and a no indicator, and the controller is adapted to proceed to a next disinfection step in the series of disinfection steps if the user selects the yes indicator, and to not proceed to the next step if the user selects the no indicator.

33. The thermal control unit of claim 22 further comprising a communication interface adapted to transmit a message to a server when the disinfection cycle has been completed, the message indicating a time when the disinfection cycle was completed.

34-48. (canceled)
